(19) United States
(12) Patent Application Publication
(10) Pub. No.: US 2024/0197655 A1
(43) Pub. Date: Jun. 20, 2024

(54) COMPOSITION COMPRISING CLOMIPHENE CITRATE AS ACTIVE INGREDIENT, FOR PREVENTING, ALLEVIATING OR TREATING NONTUBERCULOUS MYCOBACTERIA (NTM) INFECTIOUS DISEASE

(71) Applicant: REPUBLIC OF KOREA(MASAN NATIONAL TUBERCULOSIS HOSPITAL), Changwon-si, Gyeongsangnam-do (KR)

(72) Inventors: Sung Weon RYOO, Changwon-si, Gyeongsangnam-do (KR); Da Gyum LEE, Changwon-si, Gyeongsangnam-do (KR)

(73) Assignee: REPUBLIC OF KOREA(MASAN NATIONAL TUBERCULOSIS HOSPITAL), Changwon-si, Gyeongsangnam-do (KR)

(21) Appl. No.: 17/909,242

(22) PCT Filed: Feb. 22, 2022
(86) PCT No.: PCT/KR2022/002560

§ 371 Date: Sep. 2, 2022

(30) Foreign Application Priority Data
Oct. 21, 2021 (KR) 10-2021-0141086

(51) Int. Cl. A61K 31/138; A61P 31/04

(52) U.S. Cl. CPC A61K 31/138; A61P 31/04

(57) ABSTRACT

A composition for preventing, alleviating or treating nontuberculous mycobacteria (NTM) infectious diseases, contains clomiphene citrate as an active ingredient. The nontuberculous mycobacteria (NTM) are Mycobacterium abscessus (M. abscessus), Mycobacterium avium (M. avium) or Mycobacterium intracellulare (M. intracellulare). The clomiphene citrate inhibits the in-vitro growth of a wild-type strain, a clinical isolate or a clarithromycin-resistant strain of M. abscessus.

### CROSS-REFERENCE TO PRIOR APPLICATIONS

This application is a National Stage Patent Application of PCT International Patent Application No. PCT/KR2022/002560 (filed on Feb. 22, 2022) under 35 U.S.C. § 371, which claims priority to Korean Patent Application No. 10-2021-0141086 (filed on Oct. 21, 2021), which are all hereby incorporated by reference in their entirety.

### BACKGROUND

The present invention relates to a composition containing clomiphene citrate as an active ingredient to prevent, alleviate or treat nontuberculous mycobacteria (NTM) infectious diseases caused by Mycobacterium abscessus (M. abscessus), Mycobacterium avium (M. avium) or Mycobacterium intracellulare (M. intracellulare).

Nontuberculous mycobacteria (hereinafter referred to as NTM) are commonly found in the environment, including soil, natural resources and drinking water sources, to cause a high rate of human-pathogen contact. NTM infection is increasing due to a variety of host factors, such as the world's population ageing, lung diseases (including cystic fibrosis and bronchiectasis), immunosuppression, and extensive antibiotic use, which eventually causes a worldwide increase in the incidence of NTM infection.

Among NTM, Mycobacterium avium (hereinafter referred to as M. avium) and Mycobacterium abscessus (hereinafter referred to as M. abscessus) are the most common pathogens associated with lung disease. It is known that the most common pathogen causing NTM lung disease in the United States and Japan is M. avium complex that is a complex of M. avium and Mycobacterium intracellulare (hereinafter referred to as M. intracellulare). Particularly, M. abscessus is an NTM clinically important in cystic fibrosis (CF) patients. In CF patients, M. abscessus infection correlates with a faster decline in lung functions and is supposed to interfere with subsequent lung transplantation, leading to poor clinical outcomes or lifelong asymptomatic infection.

Surprisingly, the average treatment success rate for M. abscessus lung infection is no more than 45.6%, while M. abscessus is the most commonly isolated and properly growing mycobacterium. None of hitherto available treatments has proved effective in curing or long-term sputum smear conversion in patients with chronic lung disease. The current treatment recommendations for lung infections caused by M. abscessus include a combination of one or two oral antibiotics (e.g., macrolides, linezolid, clofazimine drugs, and sometimes quinolone-derived drugs) and at least two intravenous drugs (e.g., amikacin, tigecycline, imipenem, and cefoxitin).

Long-term multi-antibiotic therapy is often limited by drug-induced toxicity, including bone marrow suppression with linezolid, liver toxicity with tigecycline, and development of hypersensitivity to β-lactam. Despite rigorous therapies, the treatment failure rates remain high as the patients have recurrent or chronic symptoms. Although research studies on the experimental antibiotics and new treatments with potential activities against M. abscessus in different mechanisms are rapidly increasing, the efficacy of the antibiotics and treatments against M. abscessus has not been sufficiently studied.

Repositioning of drugs that have proven to be effective for another disease speeds up this process of drug discovery, while avoiding many of the difficulties including detailed safety data. Clomiphene citrate (hereinafter referred to as CC), known as an infertility treatment, has proven to have antibacterial activities against Gram-positive Staphylococcus aureus, Bacillus subtilis, and Enterococcus faecium. CC targets the cytoplasmic enzyme, undecaprenyl diphosphate synthase (UppS), which synthesizes the lipid carrier protein identified as undecaprenyl phosphate (Und-P) responsible for transporting a major substrate in the synthesis of wall-techoic acid (WTA).

The mechanisms of action of CC in M. abscessus are not yet known. Accordingly, the present invention is to exploit the detailed biological evaluation on CC against M. abscessus.

### SUMMARY

The present invention has been devised to solve the above problems. It is therefore an object of the present invention to provide a composition containing clomiphene citrate as an active ingredient to prevent, alleviate or treat nontuberculous mycobacteria (NTM) infectious diseases.

It is another object of the present invention to provide a health functional food composition containing clomiphene citrate as an active ingredient to prevent, alleviate or treat nontuberculous mycobacteria (NTM) infectious diseases.

The objects of the present invention are not limited to those mentioned above, and the above and other objects of the present invention will become apparent to those skilled in the art from the following description of the present invention.

The present invention is characterized by providing a composition for preventing, alleviating or treating nontuberculous mycobacteria (NTM) infectious diseases that contains clomiphene citrate as an active ingredient.

The nontuberculous mycobacteria (NTM) may be Mycobacterium abscessus (M. abscessus), Mycobacterium avium (M. avium) or Mycobacterium intracellulare (M. intracellulare).

The clomiphene citrate may inhibit the in-vitro growth of a wild-type strain, a clinical isolate or a clarithromycin-resistant strain of M. abscessus.

The present invention is also characterized by providing a health functional food composition for preventing, alleviating or treating nontuberculous mycobacteria (NTM) infectious diseases that contains clomiphene citrate as an active ingredient.

By means of solving the above problems, the present invention provides a pharmaceutical composition containing clomiphene citrate as an active ingredient to prevent, alleviate or treat nontuberculous mycobacteria (NTM) infectious diseases.

The present invention also provides an antibacterial composition against nontuberculous mycobacteria (NTM) that contains clomiphene citrate as an active ingredient.

The present invention also provides a health functional food composition containing clomiphene citrate as an active ingredient to prevent, alleviate or treat nontuberculous mycobacteria (NTM) infectious diseases.

### BRIEF DESCRIPTION OF THE DRAWINGS

FIG. 1 shows the in-vitro activity of clomiphene citrate (CC) according to an embodiment of the present invention. (A) The dose-response curves of CC against M. abscessus in 7H9 broth containing 10% OADC and cation-adjusted Mueller-Hinton (MH) broth. (B) The dose-response curves of CC against M. abscessus in medium MH broth (RFU; relative fluorescence unit). (C) The dose-response curves of CC against M. abscessus—Lux cultured under the same conditions of panel B RLUs (Relative Luciferase Units). This experimental example was performed in three biological replicates.

FIG. 2 presents the dose-response curves of CC against clinical isolates of M. abscessus according to an embodiment of the present invention. The dose-response curves were drawn from resazurin microtiter assay (REMA) data for the isolates treated with CC. The data are presented as the mean±standard deviation (SD) of triplicates for each concentration.

FIG. 3 presents the dose-response curves of CC against clarithromycin-resistant M. abscessus mutants (M. abscessus CLR-R). (A) The dose-response curves of CC against clarithromycin-resistant M. abscessus mutants (M. abscessus CLR-R). (B) A diagram showing bacterial viability evaluated using the oxidation-reduction process indicated by resazurin changing the color from blue to pink.

FIG. 4 presents the dose-response curves of CC against M. abscessus under aerobic, anaerobic or biofilm culture conditions using resazurin according to an embodiment of the present invention, showing the activity of M. abscessus under aerobic (A), anaerobic (B) or biofilm culture (C) conditions using resazurin. (A) M. abscessus was cultured to the mid-log stage, taking about 24 hours. (B) M. abscessus was cultured for 7 days in an anaerobe container system (BD GasPak™ EZ). (C) M. abscessus was assayed with a Calgary Biofilm Device (CBD).

FIG. 5 shows the effects of CC to promote the intracellular apoptosis of M. abscessus according to an embodiment of the present invention. (A) A double read analysis of CC in macrophages. Macrophages were infected with M. abscessus expressing two luciferases per cell. In 3 hours after infection, extracellular non-infectious M. abscessus were removed by washing and using gentamycin. THP-1 cells stained with SYTO 60 were counted under fluorescence detection using a Multi Reader, and the infected M. abscessus in the macrophages was counted under luminescence detection. (B) PMA-activated THP-1 was infected with M. abscessus expressing GFP for 3 hours, and non-infectious M. abscessus was removed. Subsequently, CC was applied at an indicated concentration for 3 days. Ratio sum intensity values were used in all data reduction steps. (C) The microscopic images of THP-1 cells infected with GFP-expressing M. abscessus on day 3 after treatment with the indicated concentration of CC. An automated microscope using Lionheart™ automated live cell imaging and the Gen5™ 3.05 software object feature were employed for identification of cells and M. abscessus within the imaging field. Data were expressed as mean±SD of triplicates for each concentration (scale bar=100 μm).

FIG. 6 presents the growth curves of M. abscessus under aerobic and anaerobic conditions according to an embodiment of the present invention. According to the Wayne model, M. abscessus was cultured under aerobic and anaerobic conditions. For aerobic culture, M. abscessus was cultured in 7H9 broth to the mid-log stage. In order to confirm the aerobic culture, 10 ml of the 7H9 broth was inoculated into a flask and subjected to incubation at 37° C. under spinning at 180 rpm. For anaerobic culture, M. abscessus was cultured in a GasPack container along with a substance for deoxygenating the vessel. Optical density measurement (OD600) was used for up to 96 hours at each indicated time. (∇: fading and decoloration of methylene blue as an oxygen indicator, O: conversion to aerobic culture conditions after 80 hours)

FIG. 7 shows the effects of CC on cell viability (A) and cytotoxicity (B) in human colonic epithelial cells (HCT-8) and human monocyte-macrophages (THP-In 1) according to an embodiment of the present invention. The cells were treated with CC at increasing concentrations for 24 hours. WST-8 assay (CellRix®) and CytoTox 96® Non-Radioactive Cytotoxicity Assay (Promega) were used to determine the cell viability. The cell viability measurements were acquired based on the control cells cultured in a DMSO-containing broth. Data were expressed as mean±SD of triplicates for each concentration.

FIG. 8 is a dose-response curve of CC against M. avium in 7H9 broth, showing the in-vitro activity of CC according to an embodiment of the present invention.

FIG. 9 is a dose-response curve of CC against M. intracellulare in 7H9 broth, showing the in-vitro activity of CC according to an embodiment of the present invention.

### DETAILED DESCRIPTION

Terms used in this specification will be briefly described, and the present invention will be described in detail.

The terms used in the present invention have been selected as general terms current in wide use as possible while considering their functions in the present invention, which may vary depending on the intention or precedent of those skilled in the art, the emergence of new technologies, and the like. Therefore, the term used in the present invention should be defined based on the meaning of the term and the entire contents of the present invention, rather than merely by the name of the term.

Throughout the whole specification, unless state otherwise, the term “comprises or includes” and/or “comprising or including” means that one or more other components are not excluded but further included in addition to the described components.

The embodiments of the present invention will be described below in further detail with reference to the accompanying drawings in order for those skilled in the art to implement them with ease. The present invention can be implemented in various other forms and not limited to the examples described in this specification.

Specific details including the problems to be solved for the present invention, the means for solving the problems, and the effects of the invention are included in the embodiments and drawings described below. The advantages and features of the present invention, and an achieving method thereof will become apparent with reference to the embodiments described below in detail in conjunction with the accompanying drawings.

Hereinafter, the present invention will be described in further detail with reference to the accompanying drawings.

The present invention is directed to a pharmaceutical composition for preventing, alleviating or treating nontuberculous mycobacteria (NTM) infectious diseases that contains clomiphene citrate as an active ingredient.

The nontuberculous mycobacteria (NTM) include Mycobacterium abscessus (M. abscessus), Mycobacterium avium (M. avium) or Mycobacterium intracellulare (M. intracellulare).

The clomiphene citrate inhibits the in-vitro growth of wild-type strains, clinical isolates or clarithromycin-resistant strains of Mycobacterium abscessus. The clomiphene citrate also inhibits the growth of Mycobacterium abscessus found in macrophages without cytotoxicity.

The clomiphene citrate also suppresses the growth of Mycobacterium abscessus in the non-replicating environment (i.e., anaerobic or biofilm culture) similar to the infected environment inside the human body.

Accordingly, it can be seen from the present invention that the clomiphene citrate is a potential pharmaceutical composition for treatment of infectious diseases caused by Mycobacterium abscessus, Mycobacterium avium, or Mycobacterium intracellulare.

The nontuberculous mycobacteria (NTM) infectious disease includes all the clinical symptoms caused by infection with nontuberculous mycobacteria. Examples of the NTM infectious disease may include, but are not limited to, lung disease, lymphadenitis, skin/soft tissue/bone infection, or disseminated disease.

When the composition of the present invention is a pharmaceutical composition, the pharmaceutical composition may be prepared using pharmaceutically suitable and physiologically acceptable adjuvants in addition to active ingredients. The adjuvants may include excipients, disintegrants, sweeteners, binders, coating agents, swelling agents, lubricants, flavoring agents, or solubilizers. Preferably, the pharmaceutical composition of the present invention may be formulated by further including at least one pharmaceutically acceptable carrier for the purpose of drug administration in addition to the active ingredient. In the pharmaceutical composition formulated as a liquid solution, the pharmaceutically acceptable carrier may be sterile and biocompatible, and include saline sterile water, Ringer's solution, buffered saline, albumin injection, dextrose solution, maltodextrin solution, glycerol, and ethanol, which may used alone or in combination. If necessary, the composition may further include other conventional additives, such as antioxidants, buffers, or bacteriostatic agents. Besides, the composition may further include diluents, dispersants, surfactants, binders, or lubricants to form injectable formulations (e.g., an aqueous solution, a suspension, and an emulsion), pills, capsules, granules, or tablets.

The dosage forms of the pharmaceutical composition of the present invention may include granules, powders, coated tablets, tablets, capsules, suppositories, syrups, juices, suspensions, emulsions, oils, drops, injectable solutions, sustained-release formulations of active compounds, etc. The pharmaceutical composition of the present invention may be administered in a conventional manner via intravenous, intraarterial, intraperitoneal, intramuscular, intraarterial, intraperitoneal, intrasternal, transdermal, intranasal, inhalational, topical, rectal, oral, intraocular, or intradermal routes. The effective amount of the active ingredient in the pharmaceutical composition of the present invention means an amount of the active ingredient required for prevention and/or treatment of a disease. Accordingly, it may be adjusted depending on a variety of factors, which may include, but are not limited to, the type and severity of the disease, the type and content of the active ingredient and other ingredients contained in the composition, the type of dosage form, the age, weight, general health status, sex, and diet of the patient, administration time, administration route, the secretion rate of the composition, the duration of treatment, and other drugs used along with the composition at the same time. If not limiting the present invention, when administration is given to an adult once to several times a day, for example, the composition of the present invention may be administered at a dose of 0.1 ng/kg to 10 g/kg.

The present invention is also directed to a health functional food composition for preventing, alleviating or treating nontuberculous mycobacteria (NTM) infectious diseases that contains clomiphene citrate as an active ingredient.

The nontuberculous mycobacteria (NTM) include Mycobacterium abscessus (M. abscessus), Mycobacterium avium (M. avium) or Mycobacterium intracellulare (M. intracellulare).

The clomiphene citrate inhibits the in-vitro growth of wild-type strains, clinical isolates or clarithromycin-resistant strains of Mycobacterium abscessus. The clomiphene citrate also inhibits the growth of Mycobacterium abscessus found in macrophages without cytotoxicity.

The clomiphene citrate also inhibits the growth of Mycobacterium abscessus (M. abscessus) under anaerobic (non-replicating) and biofilm culture conditions.

Preferably, the nontuberculous mycobacteria (NTM) infectious disease includes all the clinical symptoms caused by infection with nontuberculous mycobacteria. Examples of the NTM infectious disease may include, but are not limited to, lung disease, lymphadenitis, skin/soft tissue/bone infection, or disseminated disease.

When the composition of the present invention is a health functional food composition, the health functional food composition may be provided in the form of powder, granule, tablet, capsule, syrup, or beverage. The health functional food composition may include another food or food additive in addition to the active ingredient and may be used appropriately according to a conventional method. The amount of the active ingredient mixed in the composition may be suitably determined according to the intended use of the active ingredient, such as prophylactic, health, or therapeutic treatment.

The effective dose of the active ingredient in the health functional food composition may be the same as that of the active ingredient in the pharmaceutical composition. But, the health functional food composition may have a lower effective dose of the active ingredient than the pharmaceutical composition in the case of long-term use for health and hygiene or health control purpose; and even it may also have a higher effective dose of the active ingredient than the pharmaceutical composition because there is no safety issue with the active ingredient.

The type of the health food is not specifically limited and may include, for example, meat, sausage, bread, chocolate, candy, snack, confectionery, pizza, ramen and other noodles, gum, dairy products, including ice cream, soup, beverage, tea, drink, alcoholic beverage, and vitamin complex.

The following disclosure is given as to various experimental examples demonstrating the biological effects of the above composition for NTM treatment by containing clomiphene citrate (hereinafter referred to as CC). Hereinafter, a detailed description of the present invention will be given to help the understanding of the present invention with reference to the following examples, which are merely illustrative of the contents of the present invention and not intended to limit the scope of the present invention. The examples of the present invention are provided for more complete explanation of the present invention to those skilled in the art.

### EXPERIMENTAL EXAMPLES

The following experimental examples are intended to provide experimental examples commonly applied to each embodiment of the present invention.

### 1. Bacterial Strains and Culture Conditions

Three strains of M. abscessus (ATCC 19997), M. avium (ATCC 25291) and M. intracellulare (ATCC 13950) were cultured at 37° C. in Middlebrook 7H9 broth (BD, 27130) supplemented with 10% OADC (BD, 212240), 0.5% glycerol and 0.05% Tween-80. Nine clinical strains were acquired from the Korean Collection for Type Cultures (KCTC). For analysis of intracellular activity, M. abscessus strains (ATCC 19997) were used to prepare strains expressing a green fluorescent protein and luciferase. These strains were transformed by electroporation with pMV306hsp+LuxG13 (Addgene plasmid #26161) and pTEC15 (Addgene plasmid #30174).

CLR-resistant mutants of M. abscessus (ATCC 19997) were selected from 7H11 (Sigma-Aldrich, M0428) agar plates that were 25-, 50- and 100-fold higher in the minimum inhibitory concentration (MIC) to CLR than a known MIC value to CLR, 0.1 mg/L.

In order to determine the antibiotic ability against M. abscessus (ATCC 19997) under anaerobic conditions, a bacterial culture (5×10⁶ bacteria/mL) was inoculated into 5 mL of 7H9 broth (BD, 271310) supplemented with 10% OADC. The bacterial culture was placed in an anaerobic jar (BD BBLIM GasPAK™ Jar; Franklin Lakes, NJ, USA). Here, a BD BBLIM GasPAK™ anaerobic indicator (methylene blue for monitoring oxygen depletion) was used. The bacteria were cultured in cation-adjusted Mueller-Hinton broth (MH; Sigma-Aldrich, 90922) for drug susceptibility testing.

### 2. Dose-Response Curve Testing

The minimum inhibitory concentration (MIC) was determined according to the guideline by the CLSI (Clinical & Laboratory Standards Institute). The CLSI broth microdilution method recommended by MH was used with an inoculum having an OD of 0.001/ml in the exponential phase of growth. The actively growing M. abscessus, M. avium, M. intracellulare, M. abscessus-pLUX G13, CLR-R mutant, and anaerobically cultured M. abscessus were treated with the CC compound.

A serial 2-fold dilution of the CC compound was made with a 10-point serial diluent on a 96-well plate containing bacilliform bacteria in a total volume of 100 mL. Then, incubation was carried out at 37° C. for 3 hours, followed by adding 40 mL of 0.025% resazurin. After incubation overnight, the fluorescence of resorufin (metabolite resazurin) was measured with a Synergy H1 Hybrid Multi-Mode Reader (Bio-Tek, VT, USA). IC₅₀ values were calculated from raw fluorescence data using Prism 5.0 software (GraphPad Inc., La Jolla, CA, USA). Experiments were performed in triplicates.

### 3. Biofilm Assay

The first biofilm assay system was the Calgary Biofilm Device used by Bardouniotis et al. to evaluate the bactericidal activity or minimum biofilm eradication concentration (MBEC) of a biocidal active substance against Mycobacterium phlei. In the present invention, MBEC Assay® (Innovotech, Edmonton, Canada) for MBEC measurement was used. Biofilm formation was performed as follows. First, 180 mL of a bacterial inoculum (5×10⁷ bacteria/mL) was dispensed into a 96-well microliter plate, which was covered with a 96-peg lid and maintained for 5 days to form a biofilm on the pegs. 200 μL of the test solution prepared as a growth medium was added to each well of the 96-well plate. Then, the 96-well microliter plate was covered with a 96-peg lid and incubated for 4 days. After washing, the bacteria remaining on the biofilms were removed from the wells by ultrasonication in the medium. Finally, after incubation, the adherent bacteria remaining on the biofilms with or without antibiotics were quantified with resazurin.

### 4. Intracellular Apoptosis Assay

THP-1 cells were treated with phorbol 12-myristate 13-acetate (PMA; Sigma-Aldrich, USA) at a final concentration of 50 nM for 40 hours. Infection with M. abscessus containing pTEC15 green fluorescence or luciferase was performed at 37° C. in the presence of 5% CO₂ at a multiplicity of resistance (MOI) of 2:1 for 3 hours. After extensive washing with PBS, the cells were incubated in a culture medium containing 50 μg/L of gentamicin for 30 minutes and washed again with PBS. Then, 200 μL of an RPMI medium containing DMSO (negative control) and 200 μL of an RPMI medium containing CC at an indicated concentration were incubated for 3 or 4 days. Macrophages were stained with SYTO 60 (Invitrogen, USA) dye at a final concentration of 5 μM at 37° C. in a 5% CO₂ atmosphere for 30 minutes. On the day 4, luminescence was measured with a Synergy H1 microplate reader (Bio-Tek, USA). The fluorescence microscopic images of living cells were captured with a Lionheart™ FX automated microscope (BIO-Tek, USA). The Gen5™ 3.05 software object feature was available for identification of the cells in the imaging field.

### 5. Cell Viability Assay and Lactate Dehydrogenase (LDH) Cytotoxicity Assay

THP-1 (ATCC TIB-202) and HCT-8 (ATCC CCL-244) cells were measured in regards to cell viability and cytotoxicity of CC using a WST-8 cell viability assay kit (MediFab, S. Korea) and a CytoTox 96® non-radioactive cytotoxicity assay kit (Promega, Madison, WI, USA), respectively. Differentiated THP-1 cells (1.0×10⁵ cells/well) and HCT-8 cells (2.0×10⁴ cells/well) were placed in 96-well plates and incubated at 37° C. for 24 hours. Here, CC was added to the cells at different concentrations. For cell viability assay, 10 μL of the reagent (10% medium volume) was added to each well, followed by incubation for 4 hours. The resulting color was measured at 450 nm. For LDH cytotoxicity assay, 50 μL of a reagent for LDH detection was added to each well, and incubation was performed in the dark for 30 minutes. The resulting color was measured at 490 nm. In this regard, the cells treated with 1% Triton™ X-100 were used as a positive control, and those treated with DMSO were all tested negative in both experiments.

### 6. Data Analysis

Data were processed to plot graphs with a Prism version 7.0 (GraphPad) and Gen5™ 3.05 software.

### Example

1. Activity of Clomiphene Citrate (CC) Against M. abscessus

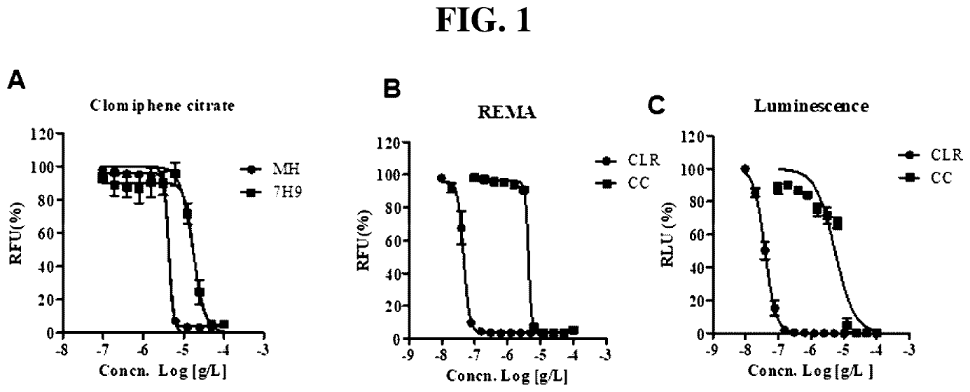

Currently, the CLSI recommends the Mueller-Hinton (MH) broth microdilution method as the gold standards for determining the MIC values of antimicrobial agents against M. abscessus. Accordingly, the Present invention acquired the MIC value of CC against M. abscessus in MH broth using resazurin rather than by adopting the 7H9 broth microdilution method. Besides, the dose-response curves of CC against M. abscessus in MH and 7H9 broths were analyzed. As shown in FIG. 1(A), the IC₅₀ of CC against M. abscessus was 4.29 mg/L in the MH broth and 17.11 mg/L in the 7H9 broth. The CC exerted similar efficacy against the reference strain of M. abscessus regardless of the type of the medium, i.e., whether the medium was MH broth or Middlebrook 7H9 broth.

The present invention employed a reporter-based assay that is suitable for drug discovery applications due to high simplicity and sensitivity relative to the dye- and absorbance-based assays. In the present invention, a luminescent reporter strain was constructed and used to determine the MIC values of CLR and CC. First, the in-vitro activities of CLR and CC were measured using the reporter-based bioluminescence, and the MIC data were acquired through the REMA method. A bioluminescent M. abscessus strain was used to verify the assay for the drug susceptibility testing. The dose-response curves of CC in M. abscessus-LuxG13 are shown in FIG. 1(C), where IC₅₀ is 5.358 mg/L. Therefore, CC is considered as an effective drug candidate against M. abscessus.

### 2. Activity of Clomiphene Citrate (CC) Against Clinical Isolates of M. Abscessus and Claritromycin-Resistant Mutant

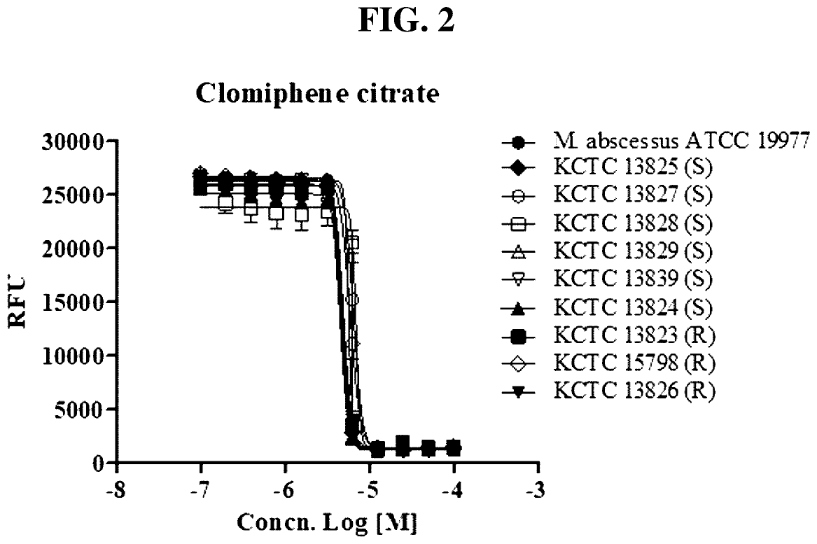

It was confirmed whether the activities of clomiphene citrate (CC) against clinical isolates of M. abscessus and a clarithromycin-resistant mutant thereof (M. abscessus CLR-R) are maintained for clinical isolate panels including rough colony (R-colony) and smooth colony (S-colony) morphologies. As shown in FIG. 2, CC was equally effective against all the nine strains of M. abscessus clinical isolate panels having an IC₅₀ value of 4.44 to 6.90 mg/L, which was similar to the MIC values observed for the subspecies of the reference strain. There was a tendency that R-colonies were far more lethal than S-colonies in a way that can resist the host defense mechanism. According to these results, CC proved to be effective in vitro against both the reference strain M abscessus (ATCC 19997) and the clinical isolates of M abscessus with R- and S-colony morphologies.

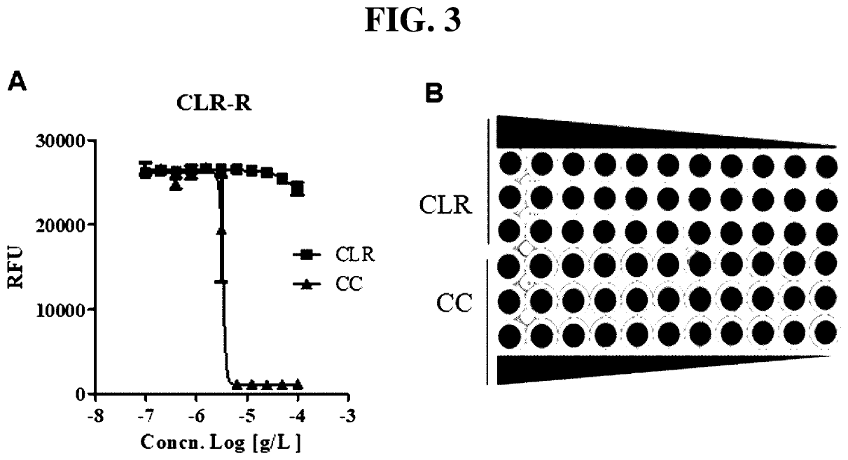

It was also tested to determine whether CC inhibits the growth of the drug-resistant strains produced in the present invention in the presence of high concentration (100 mg/L) CLR used for therapy against M. abscessus. As shown in FIG. 3, the produced resistant mutant displayed high drug resistance to CLR. Besides, CLR-resistant mutant was susceptible to CC as a wild type having a same MIC range of 3.35 to 4.439 mg/L. The results were all the same whether the mutant was resistant to or susceptible to CLR. Accordingly, CC is considered as an active drug for the treatment of CLR-resistant M. abscessus.

### 3. Effect of Clomiphene Citrate (CC) to Eradicate M. Abscessus Under Non-Replicating and Biofilm Culture Conditions

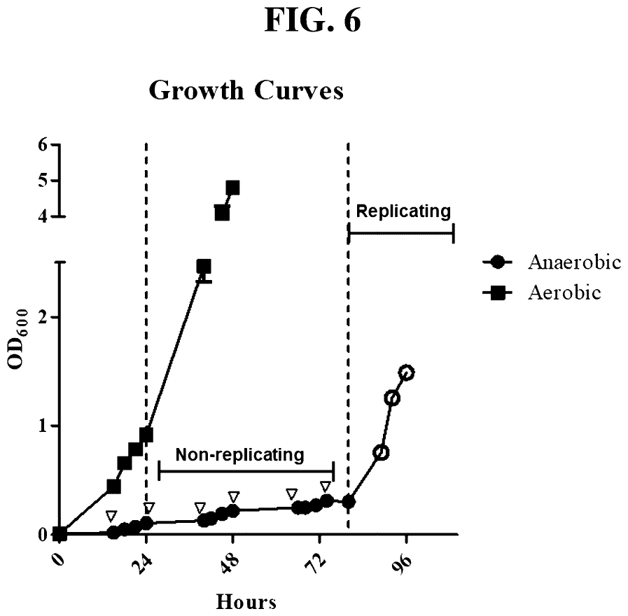

In the present invention, this step confirmed the activity of CC on the non-replicating culture induced by the depletion of oxygen. As shown in FIG. 6, the growth curves of M. abscessus under aerobic and anaerobic conditions were plotted to confirm the non-replicating conditions, prior to evaluating the effect of the drug. A comparison was made between the growth rates under aerobic, anaerobic and restored aerobic conditions in a same culture medium. It was revealed that the anaerobic conditions led to low growth rates and reduced the oxygen concentration to a level extremely low for growth. In fact, as shown in FIG. 6, decoloration was observed 15 hours after the addition of methylene blue, a dye used as an oxygen indicator, into the anaerobic container.

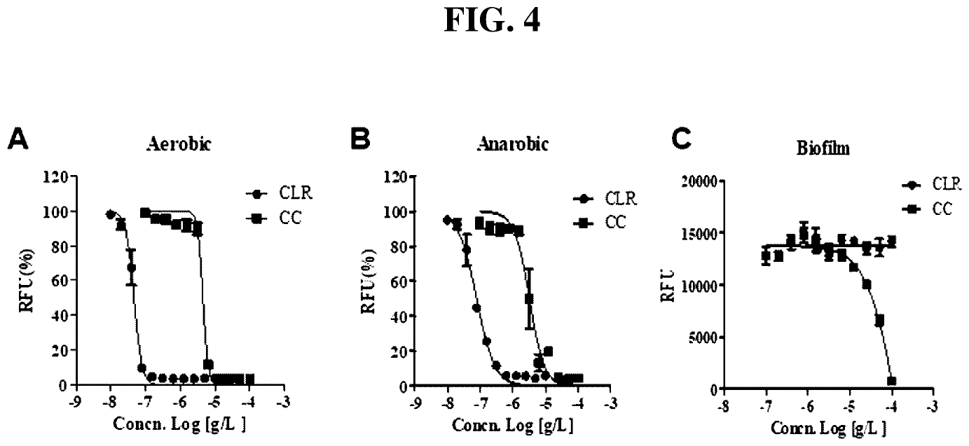

As shown in FIG. 4, CLR exhibited higher activity on the M. abscessus strains under the anaerobic culture conditions, resulting in a significant 2-fold shift of the IC₅₀ value, 77.84 μg/L, than on the M. abscessus strains under the aerobic culture conditions (IC₅₀ 46 μg/L). Interestingly, CC showed a low IC₅₀ value of 3.157 mg/L for the M. abscessus strains cultured under the anaerobic conditions, in relation to the IC₅₀ value of 4.592 mg/L for the M. abscessus strains under aerobic conditions. It was therefore confirmed that CC acquired some activity against the M. abscessus strains cultured under anaerobic non-replicating conditions. Besides, CC exerted some activity against the M. abscessus strains under anaerobic conditions, which was closely related to the non-replicating environment.

Biofilm formation is an essential factor of the antimicrobial resistance. The resistance to antibiotics, disinfectants and fungicides using biofilm-forming microbes may lead to a failure of treatment. Clinical experiences have shown that physical eradication of biofilms is necessary to the solution to infection. Munoz-Egea et al. have found a way to increase the difference between the minimum inhibitory concentration (MIC) and the minimum biofilm eliminating concentration (MBEC), especially to acquire MBEC of CLR 100,000-fold higher than MIC. However, the MBEC for M. abscessus exposed to CLR was above 100 μg, which was 2,000-fold higher than the general MIC of CLR (0.46 μg).

As shown in FIG. 4, CLR, albeit having high bactericidal activity (IC₅₀=46 μg/L) in culture, eventually lost its activity against M. abscessus in biofilm culture (IC₅₀>100 mg/L). On the flip side, CC still had an IC₅₀ of 15.94 mg/L against the bacterial in biofilm culture, showing a persistent activity against M. abscessus growing biofilms. CC also augmented the action of M. abscessus on the deoxygenated non-replicating state (IC₅₀=3.157 μg/L). These results suggest that CC is a substance of great significance in the treatment against M. abscessus.

4. Intracellular Effect of Clomiphene Citrate (CC) on M. abscessus

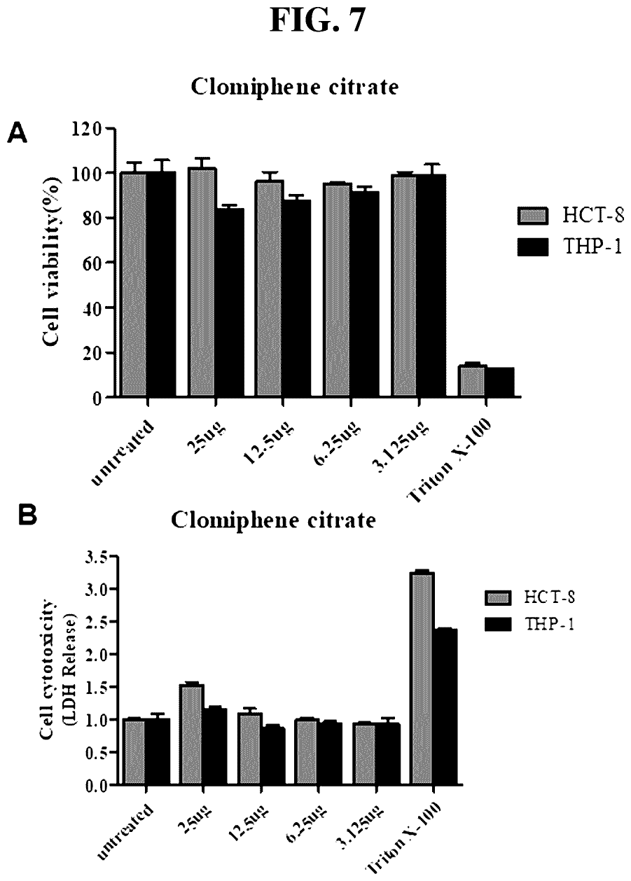

It is a common knowledge that NTMs causing lung diseases are successful facultative intracellular pathogens and surviving persisting in the host macrophages. This suggests that research protocols to search for anti-NTM drugs need to include methods of validating their effectiveness within cells. The present invention refers to the macrophage assay that involves the studies on the intracellular activity of candidate drugs against NTMs. In this regard, the cell viability at different concentrations of CC was assessed in order to determine whether CC affects the cytotoxicity. This assay was evaluated one day after treatment with CC using a Cellrix® viability assay kit and a lactate dehydrogenase (LDH) assay kit. As shown in FIG. 7, the present invention suggests that CC has no harm on the viability of human colonic epithelial cells (HCT-8) and THP-1 cells. It was observed that CC had no intracellular cytotoxicity against M. abscessus at specific concentrations.

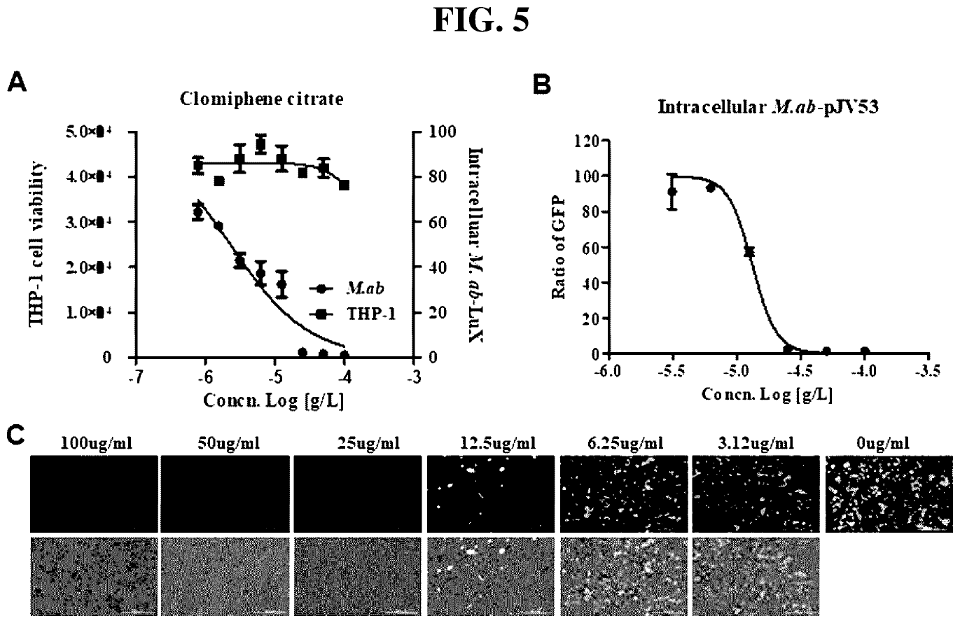

The use of a double read assay results in dose-response curves for both inhibition for M. abscessus and cytotoxicity to THP-1 cells as determined in a same experiment. Specifically, IC₅₀ and CC50 (cytotoxicity concentration inducing 50% cell death) can be determined. As shown in FIG. 5, the double dose-response curves show the response to CC over time like the data obtained from the same screening wells. The cytotoxicity was determined using a SYTO 60 probe. The treatment with CLR rescued cells from infectious attacks successfully and prevented bacterial replication (data not shown). As shown in FIG. 5(A), CC inhibited the growth of M. abscessus (lux) as indicated by an IC₅₀ of 2.294 mg/L, without cytotoxicity to the THP-1 cells, even at the highest concentration.

As shown in FIG. 5(B), as consistent with the THP-1 infection model, CC showed a similar inhibitory effect against the growth of M. abscessus-GFP in THP-1 cells as indicated by an IC₅₀ of 13.18 mg/L. This confirmed the ability of CC to reduce the intracellular infection with M. abscessus.

### 5. Activity of Clomiphene Citrate (CC) Against M. Avium

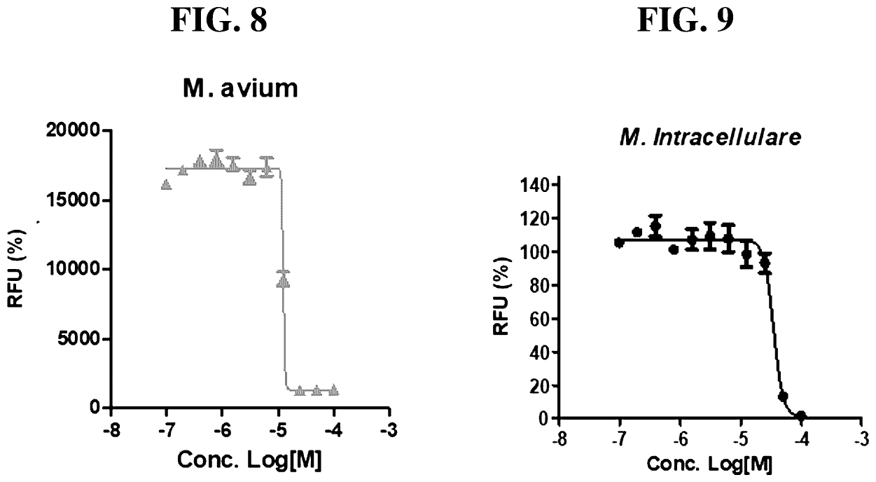

In the present invention, the resazurin microtiter assay (REMA) plate method was performed to analyze the dose-response curves of CC for M. avium in a culture medium, and then the MIC value was obtained using the Prism 5.0 program. As shown in FIG. 8, the IC₅₀ of CC against M. avium in 7H9 broth was 5.075 mg/L.

6. Activity of Clomiphene Citrate (CC) Against M. intracellulare

In the present invention, the resazurin microtiter assay (REMA) plate method was performed to analyze the dose-response curves of CC for M. intracellulare in a culture medium, and then the MIC value was obtained using the Prism 5.0 program. As shown in FIG. 9, the IC₅₀ of CC against M. intracellulare in 7H9 broth was 14.12 mg/L.

In the present invention, among other candidate chemicals with pharmacological and toxicological data, clomiphene citrate (CC), albeit a non-steroidal selective estrogen receptor modulator that stimulates ovulation in anovulatory women and blocks the estrogen receptor, proved to have a potential as a therapeutic agent for M. abscessus, M. avium or M. intracellulare infection through drug repositioning.

One of the difficulties in treating M. abscessus infection is the inherent resistance to CLR, which frequently occurs in the clinical isolates of M. abscessus, especially rough (R) morphotype clinical isolates. In order to determine whether CC is effective against strains resistant to CLR, the present invention screened spontaneous CLR-resistant mutants and confirmed that the CLR-resistant mutants had a high resistance to CLR. As shown in FIGS. 2 and 3, the in-vitro experimental results revealed that CC had activity against the CLR-resistant mutants and induced CLR-resistant and clinical R morphotype M. abscessus strains. This demonstrated that CC is quite advantageously available as a therapeutic agent for infectious diseases with M. abscessus.

The ability of M. abscessus to form biofilms represents a successful survival strategy for these ubiquitous microbes to form biofilms on the surface of airways inside the human lungs. The progression of an infectious disease caused by M. abscessus has some aspects shared with that of an infectious disease by M. tuberculosis. As the disease progresses, this pathogen survives in granulomas or lung nodules characterized by anaerobic conditions. Such a behavior of the pathogen is associated with the increased resistance to pathogenic and antimicrobial agents. Generally, drug activity is established against the active M. abscessus growing in the aerobic environment. Hence, the use of a same antibiotic against different phenotypes of bacilliform bacteria leads to different therapeutic results and often no efficacy. M. abscessus, mostly non-replicable, remains in a low metabolic status within granulomas and forms biofilms in the airways of the lungs. It is a totally different environment from the environment of normal antibiotic activity tests. In the present invention, a testing was performed on the activity of CC against M. abscessus surviving in the anaerobic and biofilm culture environments established in the lungs of patients. Advantageously, CC exhibited a great deal of activity even under the unfavorable biofilm culture conditions.

In addition, NTMs can grow and survive extracellularly and intracellularly, e.g., inside macrophages. In association with NTM lung infection, bacilliform bacteria invade the mucosa and undergo phagocytosis by macrophages. The infected macrophage cell lines may have physiological conditions mimicking the real NTM disease scene while eradicating M. abscessus. As shown in FIG. 5, CC inhibits the intracellular growth of M. abscessus in macrophage cell lines. In the present invention, it was hypothesized that CC acts as a protonophore uncoupler that destructs mycobacterial membranes and inhibits the biosynthesis of bacterial cell walls. As a result, CC proved to regulate the growth of M. abscessus.

As shown in FIGS. 8 and 9, a testing was performed to evaluate the activity of CC against M. avium and M. intracellulare in terms of IC₅₀ values that were 5.07 mg/L and 14.12 mg/L, respectively. The results showed that CC was active against M. avium and M. intracellulare and proved to have a potential as a therapeutic agent for M. avium and M. intracellulare infections.

In conclusion, the results of this study on the activity of CC claim that CC is a potent new drug against M. abscessus, M. avium and M. intracellulare infections.

By means of solving the above problems, the present invention provides a pharmaceutical composition containing clomiphene citrate as an active ingredient to prevent, alleviate or treat nontuberculous mycobacteria (NTM) infectious diseases.

The present invention also provides an antibacterial composition against nontuberculous mycobacteria (NTM) that contains clomiphene citrate as an active ingredient.

The present invention also provides a health functional food composition containing clomiphene citrate as an active ingredient to prevent, alleviate or treat nontuberculous mycobacteria (NTM) infectious diseases.

As described above, it should be apparent to those skilled in the art of the present invention that the above-described technical configuration of the present invention may be implemented in other specific forms without departing from the technical concept or essential features of the present invention.

Therefore, the foregoing examples are to be construed as merely illustrative, and not limitative of the present invention in all aspects. The scope of the present invention is defined by the appended claims rather than the foregoing detailed description and should be construed as including all changes or modifications derived from the meaning and scope of the claims and equivalents thereof.

1. A composition comprising clomiphene citrate as an active ingredient, for preventing, alleviating or treating a nontuberculous mycobacteria (NTM) infectious disease.

2. The composition according to claim 1, wherein the nontuberculous mycobacteria (NTM) are Mycobacterium abscessus.

3. The composition according to claim 1, wherein the clomiphene citrate inhibits the in-vitro growth of a wild-type strain, a clinical isolate or a clarithromycin-resistant strain of Mycobacterium abscessus.

4. The composition according to claim 1, wherein the nontuberculous mycobacteria (NTM) are Mycobacterium avium.

5. The composition according to claim 1, wherein the nontuberculous mycobacteria (NTM) are Mycobacterium intracellulare.

6. A health functional food composition comprising clomiphene citrate as an active ingredient, for preventing, alleviating or treating a nontuberculous mycobacteria (NTM) infectious disease.

7. The health functional food composition according to claim 6, wherein the nontuberculous mycobacteria (NTM) are Mycobacterium abscessus.

8. The health functional food composition according to claim 6, wherein the clomiphene citrate inhibits the in-vitro growth of a wild-type strain, a clinical isolate or a clarithromycin-resistant strain of Mycobacterium abscessus.

9. The health functional food composition according to claim 6, wherein the nontuberculous mycobacteria (NTM) are Mycobacterium avium.

10. The health functional food composition according to claim 6, wherein the nontuberculous mycobacteria (NTM) are Mycobacterium intracellulare.
